(19) United States
(12) Patent Application Publication
(10) Pub. No.: US 2023/0114035 A1
(43) Pub. Date: Apr. 13, 2023

(54) ANTIMICROBIAL COMPOSITION

(71) Applicant: EXCELSIA TECHNOLOGIES SDN BHD, Petaling Jaya (MY)

(72) Inventor: Ban Ian SIA, SELANGOR (MY)

(21) Appl. No.: 17/212,368

(22) PCT Filed: Oct. 23, 2017
(86) PCT No.: PCT/MY2017/000035

§ 371 Date: Mar. 25, 2021

(51) Int. Cl. A01N 65/36; A01N 65/22; A01N 65/26; A01N 65/28; A01N 65/44; A01N 25/02; A01N 65/42; A01N 65/40; A01P 1/00

(52) U.S. Cl. CPC A01N 65/36; A01N 65/22; A01N 65/26; A01N 65/28; A01N 65/44; A01N 25/02; A01N 65/42; A01N 65/40; A01P 1/00

(57) ABSTRACT

An antimicrobial composition comprising extracts from a plurality of plants is provided. The plants are selected from the group consisting of woody plants; succulent plants; and/or grasses. The plurality of plants may include each of a woody plant; a succulent plant; and a grass. Also provided is a method of manufacturing an antimicrobial composition, the method including the step of combining extracts from a plurality of plants selected from the group consisting of woody plants, succulent plants, and/or grasses. A method of controlling a microorganism including the step of contacting the microorganism with the antimicrobial composition is further provided. The microorganism may be located within an air conditioning system.

### INCORPORATION BY REFERENCE

This national application claims the benefit of priority under 35 U.S.C. 365 to the filing date of the International Application No. PCT/MY2017/000035, entitled “ANTIMICROBIAL COMPOSITION,” which was filed on Oct. 23, 2017, and which is incorporated herein by reference in its entirety.

### TECHNICAL FIELD

THIS invention relates to an antimicrobial composition. More particularly, the invention relates to a composition comprising plant extracts suitable for control of microorganisms such as bacterial and/or fungal agents. The invention also relates to methods of manufacture and use of the composition.

### BACKGROUND

Control of microorganisms is desirable in a variety of circumstances. For example, numerous disease and/or allergy-causing microorganisms are known. Such microorganisms may be transferred from subject to subject and/or subject to environment. Accumulation of microorganisms can also potentially soil food, damage property, and produce unwanted odours.

Air conditioning systems are one source of accumulation and spread of unwanted microorganisms. Contemporary buildings and vehicles frequently include air conditioning systems to maintain temperature at a level comfortable for human subjects. Typically, air condition systems have surfaces wherein microbes can reside, accumulate, and/or propagate. Such surfaces can include vents and filters, which may feature relatively large surface area and/or conditions (such as moisture) that can promote accumulation and/or propagation of microbes. Furthermore, movement of air within air conditioning systems can encourage the airborne distribution of microorganisms.

Various agents and compositions for control of microorganisms exist. However, existing antimicrobial agents typically have disadvantages, at least with respect to use in certain contexts. Some antimicrobial agents may have adverse effects on human or animal subjects; for example, chemical agents may be effective to control microbes, but may have substantial toxicity towards subjects when in contact with the skin or airways. Furthermore, there may issues associated with accumulated resistance of microbes to some antimicrobial agents, particularly conventional antibiotic drugs.

Another disadvantage of the use of certain existing antimicrobial agents and/or delivery methods thereof is that it may be difficult to deposit active concentrations to an area, such as of the environment, wherein microbial control is required. For example, it can be particularly difficult to deliver an effective quantity or concentration of antimicrobial agents to air conditioning systems including ducts, vents and/or filters thereof.

### SUMMARY

This invention broadly provides a composition suitable for controlling microbes, the composition comprising a plurality of plant extracts. A method of manufacturing a composition suitable for controlling microbes, the method including the step of combining a plurality of plant extracts, is also provided. The invention further broadly provides a method of controlling microorganisms, the method including the step of contacting microorganisms with a composition comprising a plurality of plant extracts.

In a first aspect, there is provided an antimicrobial composition comprising extracts from a plurality of plants, wherein the plants are selected from the group consisting of woody plants; succulent plants; and/or grasses. Preferably, the plurality of plants includes two or more of, more preferably each of, a woody plant; a succulent plant; and a grass.

In preferred embodiments, the plant extracts of the antimicrobial composition of this aspect include extracts from one or more plant parts selected from the group consisting of leaves; roots; flowers; and fruit. In particularly preferred embodiments, the plant extracts include at least one extract from a leaf; at least one extract from a root; at least one extract from a flower; and at least one extract from a fruit.

Preferably, the antimicrobial composition of this aspect is a liquid composition. In particularly preferred embodiments, the composition is an aqueous composition.

It is particular preferred according to this aspect that the plant extracts are at a concentration of between about 0.1 and about 20 w/w %.

Preferably, the antimicrobial composition includes at least one extract from a woody plant. Preferably, the woody plant is a tree or a woody herb. In certain particularly preferred embodiments, the antimicrobial composition includes at least one extract from a tree; and at least one extract from a woody herb.

Preferably, the antimicrobial composition of this aspect includes at least one extract from a woody plant of a family selected from the group consisting of Rutaceae; Lamiaceae; Meliaceae; and Myrtaceae. In a particularly preferred embodiment, the antimicrobial composition includes at least one extract from a woody plant of each of said families.

Preferably, the antimicrobial composition of this aspect includes extracts from a plurality woody plants that are trees. Preferably, the trees are of one or more genera selected from the group consisting of Citrus; Rosmarinus; Azadirachta; and Melaleuca. More preferably, the trees are of one or more species selected from the group consisting of: Citrus×aurantiifolia; Rosmarinus officinalis; Azadirachta indica; and Melaleuca alternifolia. In particularly preferred embodiments, the plurality of trees includes at least one tree of each of said species and/or genera. Preferably, the extracts are leaf extracts and/or fruit extracts.

Preferably, the composition includes at least one extract of a woody herb of the genus Thymus. More preferably, the herb is of the species Thymus zygis. Preferably, the extract is a flower extract.

Preferably, the composition includes at least one extract of a woody herb of the genus Salvia. More preferably, the woody herb is of the species Salvia officinalis. Preferably, the extract is a leaf extract.

In preferred embodiments, the composition includes an extract from at least one succulent plant. Preferably, the succulent plant is or includes a plant of the family Asphodelaceae. More preferably, the succulent plant is of the genus Aloe. Even more preferably, the succulent plant is of the species Aloe barbadensis. Preferably, the extract is a leaf extract.

In preferred embodiments, the composition includes an extract from at least one grass. Preferably, the grass is a bunchgrass. Preferably, the grass is of the genus Vetiveria.

More preferably, the grass is of the species Vetiveria zizanioides. Preferably, the extract is a root extract.

In certain preferred embodiments, the composition further includes a fragrance.

In a second aspect, the invention provides a method of manufacturing an antimicrobial composition, including the step of combining extracts from a plurality of plants selected from the group consisting of woody plants, succulent plants, and/or grasses, to thereby manufacture the antimicrobial composition.

In preferred embodiments of the second aspect, combining the extract includes the step of dissolving the extracts in liquid. Preferably, said liquid is water.

Preferably, the extracts are combined so as to be present in the antimicrobial composition at a concentration of between 0.1 and 20 w/w %. In these preferred embodiments, the method may include the step of adjusting the amount and/or construction of the extracts that are combined to form the antimicrobial composition. The method may additionally or alternatively include the step of adjusting a liquid into which the extracts are dissolved.

Preferably, the method of this aspect includes the further step of combining a fragrance agent with the extracts from the plurality of woody plants.

A third aspect of the invention provides an antimicrobial composition produced according to the method of the second aspect. Preferably, the antimicrobial composition is the antimicrobial composition of the first aspect.

In a fourth aspect, the invention provides a method of controlling a microorganism, including the step of contacting a microorganism with the antimicrobial composition of the first or third aspects, to thereby control the microorganism.

Preferably, the microorganism is a bacterial or a fungal microorganism. In some embodiments, the fungal microorganism is a yeast. In a particularly preferred embodiment, the bacterial microorganism is Staphylococcus aureus or Pseudomonas aeruginosa. In a particularly preferred embodiment, the yeast is Candida albicans.

In embodiments, the microorganism that is controlled is located on an environmental surface. Additionally or alternatively, the microorganism may be airborne.

In embodiments, the antimicrobial composition is dispersed into air to facilitate contact of the antimicrobial composition with the microorganism. In preferred such embodiments, the antimicrobial composition is dispersed as a mist, steam, or fog, to facilitate contact of the antimicrobial composition with the microorganism.

In certain particularly preferred embodiments of this aspect, the microorganism is located within an air conditioning system. Preferably, the antimicrobial composition is dispersed as a mist, steam, or fog within the air conditioning system, to contact and control a microorganism located on a surface within the air conditioning system and/or airborne within the air conditioning system.

It will be appreciated that the indefinite articles “a” and “an” are not to be read as singular indefinite articles or as otherwise excluding more than one or more than a single subject to which the indefinite article refers. For example, “a” plant includes one plant, one or more plants or a plurality of plants.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

FIG. 1 sets forth a detailed schematic of a preferred process for manufacturing an antimicrobial composition according to the invention.

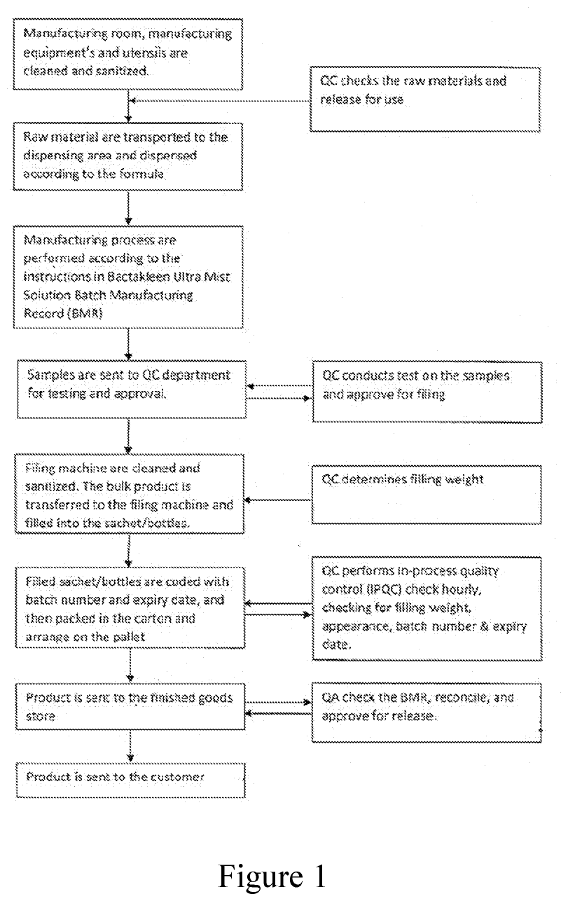

FIG. 2 sets forth a flow chart of the manufacturing process set forth in FIG. 1.

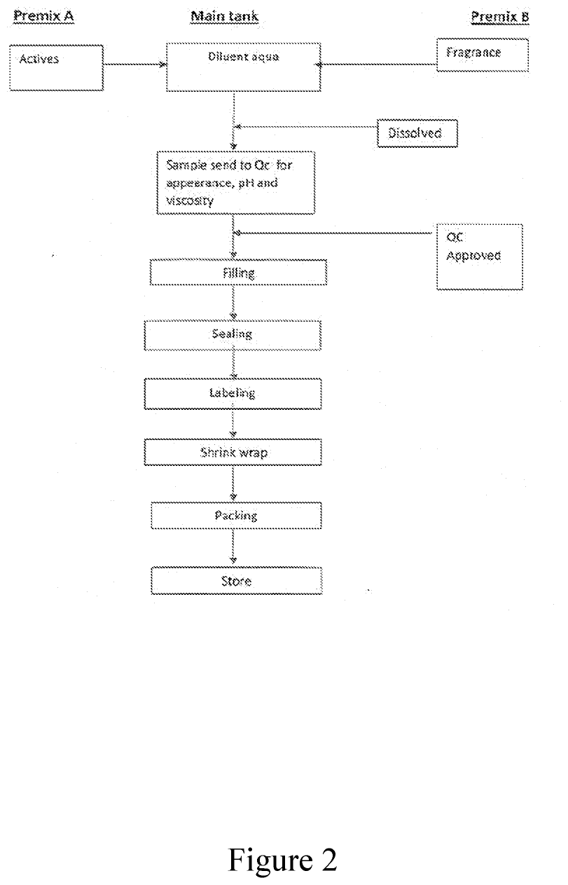

### DETAILED DESCRIPTION

The present invention is at least partly predicated on the identification of combinations of plant extracts that may be particularly effective for the control of microorganisms.

As used herein, a “plant extract” broadly refers to any substance, chemical, or agent, or combination of substances, chemicals, or agents, that is removed from a plant cell or tissue. Plant extracts may be obtained from any suitable tissue for the purposes of this invention, including structural tissues such as roots, stem, branches, and leaves; and reproductive tissues such as flowers, fruit, and seeds. A range of methods for obtaining plant extracts are known in the art. These include simple methods such as physical disruption (e.g. fruit juicing) and more involved methods that may involve multiple extraction steps (e.g. solvent treatments).

For a non-limiting overview of plant extraction techniques, the skilled person is directed to Phytochemical Methods A Guide to Modern Techniques of Plant Analysis, J. B. Harborne, Chapman & Hall (1998), incorporated herein by reference. The skilled person is further directed to Huie C. W. (2002) A Review of Modern Sample Preparation Techniques for the Extraction and Analysis of Medicinal Plants, Analytical and Bioanalytical Chemistry 373, 23-30 (incorporated herein by reference) for potentially suitable extraction techniques. It is preferred that the plant extract is obtained using a liquid-liquid extraction method. As will be known to those skilled in the art, liquid-liquid extraction methods typically involve separation using water and one or more suitable organic solvents, although without limitation thereto. For an overview of liquid-liquid extraction methods, the skilled person is directed to Recent Advances in Liquid-Liquid Extraction, C. Hanson, Elsevier Ltd (1971), incorporated herein by reference.

As used herein a “microorganism” broadly refers to organisms or organism-like biological agents that are ‘microscopic’, or too small to be seen without magnification. Microorganisms will be understood to include bacteria and microscopic fungi and algae. Furthermore, other microscopic organism-like agents capable of replication e.g. viruses and prions, will also be considered within the scope of microorganisms for the purposes of this invention.

As used herein “control” of microorganisms will be understood to refer to inhibition or prevention of growth and/or reproduction or propagation of microorganisms. The skilled person will readily understand that such control of microorganisms can include, but is not limited to, killing of microorganisms. It will be further understood that, as used herein, an agent, such as a plant extract, that is suitable for control of a microorganism, will be considered to have activity against that microorganism.

A range of methods for determining whether an agent or composition, such as a plant extract or a composition comprising same, are capable of controlling or have activity against a microorganism are known to those skilled in the art. By way of non-limiting example, the skilled person is directed to ‘Chapter 8: Methods for Testing the Antimicrobial Activity of Extracts’ in Modern Phytomedicine, I. Ahmad, F. Aqil, M. Owais (Eds) 2006.

For the purposes of this invention, any agent or composition that is capable of controlling or has activity against a microorganism is considered “antimicrobial”.

### Antimicrobial Compositions

In one aspect, the present invention provides an antimicrobial composition comprising extracts from a plurality of plants, wherein the plants are selected from the group consisting of woody plants; succulent plants; and/or grasses.

### Woody Plants

A “woody plant” will be understood to generally include plants that produce woody structural tissue. For the purposes of this invention, woody plants include, without limitation, trees, shrubs, lianas, and woody herbs. In particular regard to woody herbs, as is known in the art, certain herbs do not have bark, but can form hard stems with vascular tissues. Other herbs feature a woody stem referred to as a ‘caudex’. Both such herb types are considered woody herbs for the purpose of this invention.

The antimicrobial composition of this aspect will typically include at least one extract from a woody plant of a family selected from the group consisting of Rutaceae; Lamiaceae; Meliaceae; and Myrtaceae.

The Rutaceae family is commonly known as the citrus family, and includes fruit plants such as Citrus natsudaidai (amanatsu); Citrus medica (balady citron); Citrus bergamia (bergamot orange); Citrus×aurantium (bitter orange); Citrus×sinensis (blood orange); Citrus medica var. sarcodactylis (Buddha's hand);×Citrofortunella mitis (calamondin); Citrus reticulata×maxima (cam sánh); Citrus medica (citron); Citrus reticulate (clementine); Citrus glauca (desert lime); Citrus australasica (finger lime); Citrus×paradise (grapefruit); Citrus hystrix (kaffir lime); Citrus aurantiifolia (key lime); Citrus japonica (kumquat); Citrus limon (lemon); Citrus reticulata (mandarin); Citrus×sinensis (orange); Citrus×latifolia (Persian lime); Citrus maxima (Pomelo); Citrus australis (round lime); Citrus limetta (sweet lemon); and C. reticulata×C. maxima (tangelo).

In embodiments, the antimicrobial composition includes one or more extracts of a fruit plant of the Rutaceae family. Preferably, the fruit plant is a lime, including desert lime; finger lime; kaffir lime; and key lime. In one particularly preferred embodiment, the extract is of Citrus aurantiifolia. Typically, the extract of the citrus fruit plant will be a fruit extract, such as fruit juice.

The Lamiaceae family is commonly known as the mint or deadnettle family, and includes woody herbs or shrubs such as Lavandula angustifolia (lavender); Rosmarinus officinalis (rosemary); Origanum vulgare (oregano); Salvia officinalis (sage); and Thymus spp. (thyme).

In embodiments, the antimicrobial composition includes one or more extracts of a woody herb of the Lamiaceae family. Preferably, the extract is from leaves and/or flowers of the woody herb. Preferably, the one or more extracts are of sage, rosemary and/or thyme. In a particularly preferred embodiment, the composition includes an extract of Salvia officinalis, an extract of Rosmarinus officinalis, and an extract of Thymus zygis. In embodiments wherein the composition includes an extract of Salvia officinalis and/or Rosmarinus officinalis, typically the extract will be a leaf extract. In embodiments wherein the composition includes an extract of Thymus zygis, typically the extract will be a flower extract.

The Meliaceae family is commonly known as the mahogany family, and includes trees such as Azadirachta indica (neem tree); Carapa procera (crabwood); Cedrela odorata (Spanish cedar); Guarea guidonia (grape bark); and Melia azedarach (chinaberry).

In embodiments, the antimicrobial composition includes one or more extracts of a tree of the Meliaceae family. In a particularly preferred embodiment, the composition includes an extract of Azadirachta indica. Typically, the extract of the tree of the Meliaceae family will be a leaf extract.

The Myrtaceae family is commonly known as the myrtle family, and includes trees and shrubs in a broad range of genera, including Melaleuca. The Melaleuca genus includes trees commonly known as paperbarks, honey myrtles, and tea trees.

In embodiments, the antimicrobial composition includes one or more extracts of a tree of the Myrtaceae family, preferably of the Melaleuca genera. In a particularly preferred embodiment, the composition includes an extract of tea tree, preferably Melaleuca alternifolia. Typically, the extract of the tree Myrtaceae family is an extract of the leaves of the tree.

### Succulent Plants

A “succulent plant” or “succulent” will be understood to generally include plants with relatively ‘thick’, ‘fleshy’ or ‘swollen’ stems and/or leaves, adapted for dry environments.

The antimicrobial composition of this aspect will typically include at least one extract from a succulent plant of the Asphodelaceae family. Preferably, the extract is of a succulent plant of the Aloe genus. In a particularly preferred embodiment, the extract is of Aloe barbadensis (alternatively referred to as Aloe vera). Typically, the extract of the succulent plant of the Asphodelaceae family is an extract of the leaves of the succulent plant.

### Grasses

A “grass” will be understood to generally include plants of the Poaceae or Gramineae family.

The antimicrobial composition of this aspect will typically include at least one extract from a grass. Preferably the grass is of the genus Chrysopogon or Vetiveria. In embodiments, the grass is a bunchgrass or tussock grass. As used herein, “bunchgrass” and “tussock grass” will be understood to refer to grass that typically grows as a single or small number of plants in bunches or ‘clumps’, in contrast to forming a lawn or turf. Preferably the grass is a vetiver grass, preferably of the species Chrysopogon zizanioides or Vetiveria zizanioides. Typically, the extract of the grass is an extract of the roots of the grass.

Typically, plant extracts of the composition of this aspect are present in the antimicrobial composition at a concentration of between about 0.1 and about 20 w/w % including about: 0.12, 0.14, 0.16, 0.18, 0.2, 0.22, 0.24, 0.25, 0.26, 0.28, 0.3, 0.32, 0.34, 0.36, 0.38, 0.4, 0.42, 0.44, 0.46, 0.48, 0.5, 1, 2, 3, 4, 5, 6, 7, 8, 9, 10, 11, 12, 13, 14, 15, 16, 17, 18, and 19 w/w %.

In embodiments wherein the composition comprises an extract from a woody plant of the Rutaceae family, typically the extract is at a concentration of about 5 to about 20 w/w %, including about: 6, 7, 8, 9, 10, 11, 12, 13, 14, 15, 16, 17, 18, and 19 w/w %.

In embodiments wherein the composition comprises an extract from a woody plant of the Lamiaceae family, typically the extract is at a concentration of about 0.1 to about 5 w/w %, including about: 0.1, 0.25, 0.75, 1, 1.25, 1.5, 1.75, 2, 2.25, 2.5, 2.75, 3, 3.25. 3.5, 3.75, 4, 4.25, 4.5, and 4.75 w/w %.

In embodiments wherein the composition comprises an extract from a woody plant of the Meliaceae family, typically the extract is at a concentration of about typically the extract is at a concentration of about 0.1 to about 5 w/w %, including about: 0.1, 0.25, 0.75, 1, 1.25, 1.5, 1.75, 2, 2.25, 2.5, 2.75, 3, 3.25. 3.5, 3.75, 4, 4.25, 4.5, and 4.75 w/w %.

In embodiments wherein the composition comprises an extract from a woody plant of the Myrtaceae family, typically the extract is at a concentration of about 1 to about 10 w/w %, including about: 1.5, 2, 2.5, 3, 3.5, 4, 4.5, 5, 5.5, 6, 6.5, 7, 7.5, 8, 8.5, 9, and 9.5 w/w %.

In embodiments wherein the composition comprises an extract from a succulent plant of the Asphodelaceae family, typically the extract is at a concentration of about 0.1 to about 5 w/w %, including about: 0.1, 0.25, 0.75, 1, 1.25, 1.5, 1.75, 2, 2.25, 2.5, 2.75, 3, 3.25. 3.5, 3.75, 4, 4.25, 4.5, and 4.75 w/w %.

In embodiments wherein the composition comprises an extract from a grass, typically the extract is at a concentration of about 0.1 to about 5 w/w %, including about: 0.1, 0.25, 0.75, 1, 1.25, 1.5, 1.75, 2, 2.25, 2.5, 2.75, 3, 3.25. 3.5, 3.75, 4, 4.25, 4.5, and 4.75 w/w %.

Typically, the antimicrobial composition of this aspect is a liquid composition. In embodiments wherein the composition is a liquid composition, the liquid composition may comprise any suitable solvent, including non-polar solvents such as pentane, hexane, cyclohexane, benzene, toluene, and diethyl ether; ‘intermediate’ or semi-polar solvents such as dichloromethane and ethylacetate; polar protic solvents such as ammonia, t-butanol, n-propanol, ethanol, methanol, acetic acid, and water; and polar aprotic solvents such as acetone, and dimethyl sulfoxide.

Typically, in embodiments wherein the composition of this aspect is a liquid composition, the composition is an “aqueous” composition. As used herein, an aqueous composition will be understood to refer to a composition wherein the primary solvent is water.

In embodiments, the antimicrobial composition of this aspect includes a fragrance agent, substance, or chemical. As used herein a “fragrance agent” and the like will be understood to refer to an agent added to the composition for the purposes of changing, adjusting, or modifying the fragrance, smell, or aroma of the composition. Typically, the fragrance agent will be selected to be desirable, or at least substantially inoffensive, to a subject, such as a human subject. The fragrance agent need not, but may, have antimicrobial activity.

In embodiments, the antimicrobial composition according to this aspect has low, limited, or substantially absent toxicity and/or allergenicity or allergenic potential to a subject, such as a human subject. Techniques for assessing toxicity and allergenicity are well known in the art. By way of non-limiting example, the skilled person is directed to Parasuraman, S. (2011) J. Pharmacol. Pharmacother. 2(2): 74-79; and Oppenheimer, J. et. al. (2014) Allergic Diagnostic Testing, World Allergy Organization, July 2014

<http://www.worldallergy.org/professional/allergic_diseases_center/allergy_diagnostic/>, incorporated herein by reference.

### Manufacture of Antimicrobial Compositions

Another aspect of the invention provides a method of manufacturing an antimicrobial composition, including the step of combining extracts from a plurality of plants selected from the group consisting of woody plants, succulent plants, and/or grasses. The antimicrobial composition manufactured according to the method of this aspect will suitably be as hereinabove described for the preceding aspect.

Typically, the method of this aspect includes the step of dissolving the extracts of the plurality of plants in a solvent. In embodiments, the method may include the step of combining a fragrance agent with the plant extracts and/or solvent.

The method of manufacturing the antimicrobial composition may include one or more of the following steps:
(a) cleaning and sterilisation of equipment for manufacture;
(b) conducting initial quality control of materials (e.g. plant extracts, solvents, and/or fragrances);
(c) mixing of the plant extracts with a solvent;
(d) mixing of a fragrance agent with the plant extracts and/or a solvent
(d) quality control checks of the formed composition comprising plant extracts, and optionally a solvent and/or fragrance;
(e) cleaning and sterilisation of dispensing equipment for dispensing the composition
(f) cleaning and sterilisation of containers for container the composition (e.g. sachets or bottles);
(g) ‘in process’ quality control checks of the dispensed composition (e.g. for filing weight, appearance, batch number and/or expiry);
(h) storage of the final product in the form of the composition in a sealed container;
(i) quality assurance assessment and approval of the final product for release to consumers.

In embodiments wherein the method includes steps (c) and (d) above, typically, mixing of the plant extracts with the solvent as per step (c) is performed in a first chamber, tank, or container; and mixing of the fragrance agent with the solvent as per step (d) is performed in a second chamber, tank, or container; and the mixed or ‘premixed’ extract-solvent and fragrance-solvent solutions are subsequently combined in a third chamber, tank, or container.

### Control of Microorganisms

In a further aspect, the invention provides a method of controlling a microorganism including the step of contacting a microorganism with an antimicrobial composition as described herein with.

The microorganism that is controlled according to this aspect may be any suitable microorganism. Typically, the microorganism will be a bacteria, fungi, or virus.

The bacterial microorganism may be a gram negative bacteria or a gram positive bacteria. The bacterial microorganism may be a coccus, bacillus, vibrio, spirillum, or spirochete. In some preferred embodiments, the bacterial microorganism is Staphylococcus, preferably S. aureus. In some preferred embodiments, the bacterial microorganism is Pseudomonas, preferably P. aeruginosa.

The fungi may be a yeast. In some preferred embodiment, the fungi is Candida, preferably C. albicans.

The microorganism that is controlled according to the method may be located on an environmental surface, or may be airborne. Typically, the microorganism will be located in or on an artificial or ‘human-made’ entity. Said entity may be a vehicle, such as a car; a bus; a train; a monorail; a boat; a ship; an aeroplane; a helicopter; a construction vehicle (e.g. a crane, bulldozer, or excavator); an agricultural vehicle (e.g. a tractor or harvestor); or a military vehicle (e.g. a tank or personnel carrier). Said entity may be a building, such as an agricultural building (e.g. an abattoir, greenhouse, or farmhouse); a commercial building (e.g. a supermarket, warehouse, or shopping centre); a residential building (e.g. a house, apartment block, or dormitory); a medical building (e.g. a hospital or nursing home); an educational building (e.g. a school, library, or college); a government building (e.g. a consulate, embassy, or hall); an industrial building (e.g. a factory or power plant); a military building (e.g. barracks or a bunker); a religious building (e.g. a church, mosque, or synagogue); or a transport building (e.g. an airport, bus station, or subway).

It is particularly preferred that the microorganism that is controlled according to the method of this aspect is located within an air conditioning system. As will be known to the skilled person, air conditioning systems may take several forms, including ‘window unit’ systems; ‘split systems’ including ductless e.g. ‘mini-split’ systems, and ducted or ‘central’ systems; and portable systems.

In some preferred embodiments wherein the microorganism that is controlled is located within an air conditioning system, the air conditioning system will be a ducted or central air condition system. As will be known to those skilled in the art, ducted air conditioning systems distribute conditioned air through tubes or passageways into a structure, such as a building or vehicle, through openings or vents. The microorganism that is controlled within a ducted air conditioning system may be located on a surface of the system, such as the interior or a duct, a vent, or a filter, or may be airborne within the system.

In embodiments of this aspect, the microorganism is contacted with the composition wherein the composition is dispersed into the air or otherwise delivered or distributed to the location of the microorganism in gaseous and/or airborne form, for example as a steam, mist, or fog. It will be appreciated that a liquid composition of the preceding aspects may be converted, modified, or changed into a gaseous and/or airborne form for delivery of the composition to a location of the microorganism by a range of suitable processes. Equipment for generation of steam, fog, and/or mist using water and/or aqueous solutions is known in the art. Such equipment includes steam cleaning apparatus; and logger' apparatus, sometimes referred to as ‘mould foggers’.

The delivery or distribution of compositions in gaseous and/or airborne form is considered particularly advantageous according to this aspect, especially in the context of control of microorganisms located within an air conditioning system such as a ducted air conditioning system. It has been realised that said delivery may be particularly effective for bringing suitable quantities of antimicrobial compositions into contact with microorganisms in the air and/or on surfaces that may be difficult to otherwise reach or access.

In addition to the applications set forth above, this aspect broadly encompasses methods of administration of antimicrobial compositions described herein to a subject in need thereof, to thereby treat or prevent a condition caused by or associated with the presence of a microorganism in or on the subject.

In order that the invention may be readily understood and put into practical effect, particular preferred embodiments will now be described by way of the following non-limiting example.

### EXAMPLES

### Example 1

### Formula of Preferred Antimicrobial Composition

The following optimised formula of active ingredients has been developed, and is considered especially desirable for its antimicrobial properties, particularly in the context of application to air conditioning systems.

| | | Concentration |
|---|---|---|
| Plant Extract | Extraction type | (w/w %) |
| Citrus aurantifolia | Liquid-Liquid | 12.5 |
| (key lime) juice | | |
| Rosmarinus officinalis | Liquid-Liquid | 2.5 |
| (rosemary) leaf oil | | |
| Azadirachta indica | Liquid-Liquid | 0.25 |
| (neem tree) leaf extract | | |
| Salvia officinalis | Liquid-Liquid | 0.25 |
| (sage) leaf oil | | |
| Aloe barbadensis | Liquid-Liquid | 0.25 |
| (aloe vera) leaf juice | | |
| Thymus zygis | Liquid-Liquid | 0.25 |
| (thyme) flower oil | | |
| Vetiveria zizanoides | Liquid-Liquid | 0.25 |
| (vetiver) root oil | | |
| Melaleuca alternifolia | Liquid-Liquid | 5.0 |
| (tea tree) leaf oil | | |

Throughout the specification, the aim has been to describe the preferred or typical embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

1. An antimicrobial composition comprising extracts from a plurality of plants including two or more of a woody plant a succulent plant, and a grass.

2. The composition of claim 1, wherein the extracts include one or more of a root extract, a leaf extract, a flower extract, and a fruit extract.

3. The composition of claim 1 or claim 2, wherein the extracts are at a concentration of between bout 0.1 and about 20 w/w %.

4. The composition of any preceding claim, wherein the extracts include at least one extract from a tree; and at least one extract from a woody herb.

5. The composition of any preceding claim, wherein the extracts include at least one extract from a woody plant of a family selected from the group consisting of Rutaceae; Lamiaceae; Meliaceae: and Myrtaceae.

6. The composition of claim 5, wherein the extracts include at least one extract from a woody plant of each of the families Rutaceae; Lamiaceae; Meliaceae and Myrtaceae.

7. The composition of any preceding claim, wherein the extracts include extracts from a plurality of trees.

8. The composition of claim 7, wherein the trees are of one or more genera selected from the group consisting of Citrus: Rosmarinus; Azadirachta; and Melaleuca.

9. The composition of claim 7 or claim 8, wherein the trees are of one or more species selected from the group consisting of: Citrus×aurentiifolia; Rosmarinus officinalis; Azadirachta indica; and Melaleuca alternifolia.

10. The composition of any one of claims 7-9, wherein the plurality of trees includes at least one tree of each of the genera Citrus: Rosmarinus; Azadirachta; and Melaleuca, and/or each of the species Citrus×aurantiifolia; Rosmarinus officinalis; Azadirachta indica; and Melaleuca alternifolia.

11. The composition of any one of claims 7-10, wherein the extracts are leaf extracts and/or fruit extracts.

12. The composition of any preceding claim, wherein the extracts include at least one extract of a woody herb of the genus Thymus.

13. The composition of claim 12, wherein the extract is of the species Thymus zygis.

14. The composition of claim 12 or claim 13, wherein the extract is a flower extract.

15. The composition of any preceding claim, wherein the extracts include at least one extract of a woody herb of the genus Salvia.

16. The composition of claim 15, wherein the extract is of the species Salvia officinalis.

17. The composition of claim 15 or claim 16, wherein the extract is a leaf extract.

18. The composition of any preceding claim, wherein the extracts include an extract from at least one succulent plant.

19. The composition of claim 18, wherein the extract is of a succulent plant of the family Asphodelaceae.

20. The composition of claim 19, wherein the extract is of the genus Aloe.

21. The composition of claim 20, wherein the extract is of the species Aloe barbadensis.

22. The composition of any one of claims 18-21, wherein the extract is a leaf extract.

23. The composition of any preceding claim, wherein the extracts include an extract from at least one crass.

24. The composition claim 23, wherein the grass is a bunchgrass.

25. The composition of claim 24, wherein the grass is of the genus Vetiveria.

26. The composition of claim 25, wherein the grass is of the species Vetiveria zizanioides.

27. The composition of any one of claims 23-26, wherein the extract is a root extract.

28. The composition of any preceding claim, wherein the composition further includes a fragrance.

29. A method of manufacturing an antimicrobial composition, including the step of combining extracts from a plurality of plants including two or more of a woody plant; a succulent plant and a grass, to thereby manufacture the antimicrobial composition.

30. A composition produced according to the method of claim 29.

31. The composition of claim 30, wherein the composition is of any one of claim 1-28.

32. A method of controlling a microorganism, including the step of contacting a microorganism with the antimicrobial composition of any one of claim 1-28, or 30-31 to thereby control the microorganism.

33. The method of claim 32, herein the microorganism is a bacterial or a fungal microorganism.

34. The method of claim 33, wherein the bacterial microorganism is Staphylococcus aureus or Pseudomonas aeruginosa.

35. The method of claim 33, wherein the fungal organism is Candida albicans.

36. The method of army one of claims 32-35, wherein the microorganism that is controlled is located on an environmental surface.

37. The method of any one of claims 32-35, wherein the microorganism that is controlled is airborne.

38. The method of any one of claims 32-37, wherein the antimicrobial composition is controlled by dispersing the composition into air to facilitate contact of the composition with the microorganism.

39. The method of claim 38, wherein the antimicrobial composition is dispersed as a mist, steam, or fog, to facilitate contact of the composition with the microorganism.

40. The method of any one of claims 32-39, wherein the microorganism that is controlled is located within an air conditioning system.
